(19) United States
(12) Patent Application Publication
(10) Pub. No.: US 2006/0083681 A1
(43) Pub. Date: Apr. 20, 2006

(54) Compounds for myocardial perfusion imaging

(76) Inventors: Ajay Purohit, Sudbury, MA (US); David S. Casebier, Carlisle, MA (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(21) Appl. No.: 11/243,869

(22) Filed: Oct. 5, 2005

Related U.S. Application Data
(60) Provisional application No. 60/619,678, filed on Oct. 18, 2004.

(51) Int. Cl. A61K 51/00; C07F 15/00; C07F 5/00; C07F 13/00

(52) U.S. Cl. 424/1.11; 534/11; 534/15; 540/465; 534/14; 546/2

(57) ABSTRACT

The present disclosure is directed to compounds comprising imaging moieties and their use for diagnosing certain disorders.

The present disclosure is generally directed to compounds comprising imaging moieties and their use for diagnosing certain disorders.

Mitochondria are membrane-enclosed organelles distributed through the cytosol of most eukaryotic cells and are particularly concentrated in myocardium tissue. There are a variety of enzymes found in the mitochondria that catalyze the oxidation of organic matter releasing energy in the process. One such enzyme, MC-1 (“mitochondrial complex 1” or “complex 1”), plays a major role in this process.

It has been recognized that interrupting the normal function of mitochondria (e.g., by binding to MC-1) could advantageously concentrate certain compounds in the mitochondria, and hence in the mitochondria-rich myocardium tissue. If these compounds were bound to an imaging moiety such a build-up could be detected, thereby providing valuable diagnostic markers for myocardial perfusion imaging, a technique that evaluates regional myocardial blood flow and viability under stress and rest conditions.

In one embodiment the present disclosure provides a compound of formula (I) 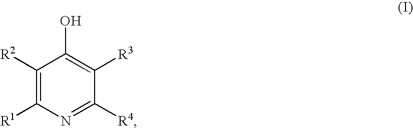
or a pharmaceutically acceptable salt thereof, wherein

R¹ and R² are alkoxy optionally substituted with an imaging moiety; or

R¹ and R², together with the carbon atoms to which they are attached, form a six-membered aromatic ring containing zero or one nitrogen atoms optionally substituted with alkoxy or an imaging moiety; wherein the alkoxy is further optionally substituted with an imaging moiety; and

R³ and R⁴ are independently alkenyl, alkyl, alkynyl, aryloxyalkyl, or arylalkyl, wherein the alkenyl, the alkyl, the alkynyl, and the alkyl part of the aryloxyalkyl and the arylalkyl are each optionally substituted with an imaging moiety, and wherein the aryl part of the aryloxyalkyl and the arylalkyl are optionally substituted with alkoxy, a second alkyl group, or an imaging moiety, wherein the alkoxy and the second alkyl group are each optionally substituted with an imaging moiety;

provided that at least one imaging moiety is present.

In another embodiment the imaging moiety is selected from a radioisotope for nuclear medicine imaging, a paramagnetic species for use in MRI imaging, an echogenic entity for use in ultrasound imaging, a fluorescent entity for use in fluorescence imaging, and a light-active entity for use in optical imaging.

In another embodiment the imaging moiety is a paramagnetic species for use in MRI imaging, wherein the paramagnetic species is selected from Gd³⁺, Fe³⁺, In³⁺, and Mn²⁺.

In another embodiment the imaging moiety is an echogenic entity for use in ultrasound imaging, wherein the echogenic entity is a fluorocarbon encapsulated surfactant microsphere.

In another embodiment the imaging moiety is a radioisotope for nuclear medicine imaging wherein the radioisotope is selected from ¹¹C, ¹³N, ¹⁸F, ¹²³I, ¹²⁵I, ^{99m}Tc, ⁹⁵Tc, ¹¹¹In, ⁶²Cu, ⁶⁴Cu, ⁶⁷Ga, and ⁶⁸Ga. In another embodiment the radioisotope is ¹⁸F. In another embodiment the radioisotope is ^{99m}Tc.

In another embodiment the present disclosure provides an imaging agent comprising a compound of formula (I) and a metal bonding unit having a formula selected from 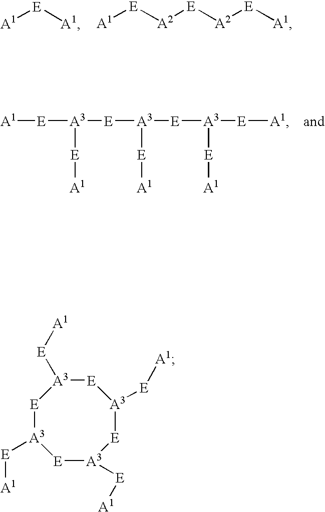
wherein

each A¹ is independently selected from a bond to the compound of formula (I), —NR⁵R⁶, —SH, —S(Pg), —OH, —PR⁵R⁶, and —P(O)R⁷R⁸;

each A² is independently selected from S, O, and PR⁵;

A³ is N;

each E is independently selected from C₁₋₁₀alkylene substituted with 0-3 R⁹ groups, C₆₋₁₀arylene substituted with 0-3 R⁹ groups, C₃₋₁₀cycloalkylene substituted with 0-3 R⁹ groups, heterocyclyl-C₁₋₁₀alkylene substituted with 0-3 R⁹ groups, C₆₋₁₀aryl-C₁₋₁₀alkylene substituted with 0-3 R⁹ groups, and heterocyclyl substituted with 0-3 R⁹ groups;

R⁵ and R⁶ are each independently selected from a bond to the compound of formula (I), C₁₋₁₀alkyl substituted with 0-3 R⁸ groups, C₆₋₁₀aryl substituted with 0-3 R⁸ groups, C₃₋₁₀cycloalkyl substituted with 0-3 R⁸ groups, heterocyclyl-C₁₋₁₀alkyl substituted with 0-3 R⁸ groups, and heterocyclyl substituted with 0-3 R⁸ groups;

R⁷ and R⁸ are each independently selected from a bond to the compound of formula (I), C₁₋₁₀alkyl substituted with 0-3 R⁹ groups, C₆₋₁₀aryl substituted with 0-3 R⁹ groups, C₃₋₁₀cycloalkyl substituted with 0-3 R⁹ groups, heterocyclyl-C₁₋₁₀alkyl substituted with 0-3 R⁹ groups, C₆₋₁₀aryl-C₁₋₁₀alkyl substituted with 0-3 R⁹ groups, heterocyclyl substituted with 0-3 R⁹ groups, and hydroxy;

each R⁹ is independently selected from a bond to the compound of formula (I), C₂₋₄alkenyl, C₁₋₆alkoxy, C₁₋₆alkoxycarbonyl, di(C₁₋₆alkyl)amino, C₁₋₆alkylcarbonyl, amino, cyano, C₃₋₆cycloalkyl, formyl, halo, haloalkoxy, haloalkyl, hydroxy, nitro, and oxo; and

Pg is a thiol protecting group;

provided at least one bond to the compound of formula (I) is present.

In another embodiment the present disclosure provides a complex comprising a compound of formula (I) and a metal bonding unit having a formula selected from 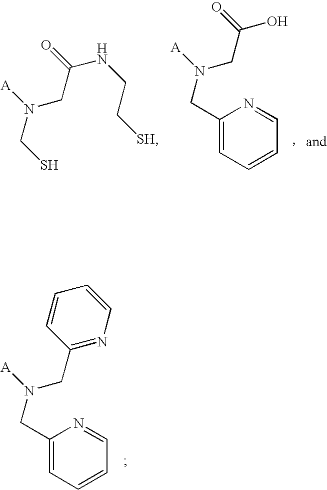
wherein A is a bond to the compound of formula (I). In another embodiment the imaging moiety is ^{99m}Tc.

In another embodiment the present disclosure provides a composition comprising a compound of formula (I) and/or a pharmaceutically acceptable salt thereof, and a pharmaceutically acceptable carrier.

In another embodiment the present disclosure provides a method of imaging myocardial perfusion in a patient, the method comprising:

(a) administering to a patient a compound of formula (I) 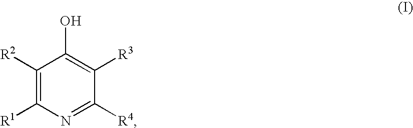
or a pharmaceutically acceptable salt thereof, wherein

R¹ and R² are alkoxy optionally substituted with an imaging moiety; or

R¹ and R², together with the carbon atoms to which they are attached, form a form a six-membered aromatic ring containing zero or one nitrogen atoms optionally substituted with alkoxy or an imaging moiety; wherein the alkoxy is further optionally substituted with an imaging moiety; and
R³ and R⁴ are independently alkenyl, alkyl, alkynyl, aryloxyalkyl, or arylalkyl, wherein the alkenyl, the alkyl, the alkynyl, and the alkyl part of the aryloxyalkyl and the arylalkyl are each optionally substituted with an imaging moiety, and wherein the aryl part of the aryloxyalkyl and the arylalkyl are optionally substituted with alkoxy, a second alkyl group, or an imaging moiety, wherein the alkoxy and the second alkyl group are each optionally substituted with an imaging moiety;

provided that at least one imaging moiety is present; and

(b) acquiring an image of a site of concentration of the compound in the patient by a diagnostic imaging technique.

In another embodiment the imaging moiety is a radioisotope for nuclear medicine imaging, a paramagnetic species for use in MRI imaging, an echogenic entity for use in ultrasound imaging, a fluorescent entity for use in fluorescence imaging, or a light-active entity for use in optical imaging.

In another embodiment the imaging moiety is a paramagnetic species for use in MRI imaging, wherein the paramagnetic species is selected from Gd³⁺, Fe³⁺, In³⁺, and Mn²⁺.

In another embodiment the imaging moiety is an echogenic entity for use in ultrasound imaging, wherein the echogenic entity is a fluorocarbon encapsulated surfactant microsphere.

In another embodiment the imaging moiety is a radioisotope for nuclear medicine imaging wherein the radioisotope is selected from ¹¹C, ¹³N, ¹⁸F, ¹²³I, ¹²⁵I, ^{99m}Tc, ⁹⁵Tc, ¹¹¹In, ⁶²Cu, ⁶⁴Cu, ⁶⁷Ga, and ⁶⁸Ga. In another embodiment the radioisotope is ¹⁸F. In another embodiment the radioisotope is ^{99m}Tc.

All patents, patent applications, and literature references cited in the specification are herein incorporated by reference in their entirety. In the case of inconsistencies, the present disclosure, including definitions, will prevail.

As used in the present specification, the following terms have the meanings indicated:

As used herein, the singular forms “a”, “an”, and “the” include plural reference unless the context clearly dictates otherwise.

The number of carbon atoms in any particular group is denoted before the recitation of the group. For example, the term “C₆₋₁₀arylene” denotes an arylene group containing from six to ten carbon atoms, and the term “C₆₋₁₀aryl-C₁₋₁₀alkylene,” refers to an aryl group of six to ten carbon atoms attached to an alkylene group of one to ten carbon atoms.

The term “alkenyl,” as used herein, refers to a straight or branched chain group of two to sixteen carbon atoms containing at least one carbon-carbon double bond.

The term “alkoxy,” as used herein, refers to an alkyl group attached to the parent molecular moiety through an oxygen atom.

The term “alkoxycarbonyl,” as used herein, refers to an alkoxy group attached to the parent molecular moiety through a carbonyl group.

The term “alkyl,” as used herein, refers to a group derived from a straight or branched chain saturated hydrocarbon containing from one to sixteen carbon atoms.

The term “alkylcarbonyl,” as used herein, refers to an alkyl group attached to the parent molecular moiety through a carbonyl group.

The term “alkylene,” as used herein, refers to a divalent group of two to sixteen carbon atoms derived from a straight or branched chain saturated hydrocarbon.

The term “alkynyl,” as used herein, refers to a straight or branched chain hydrocarbon of two to sixteen carbon atoms containing at least one carbon-carbon triple bond.

The term “amino,” as used herein, refers to —NH₂.

The term “aryl,” as used herein, refers to a phenyl group, or a bicyclic fused ring system wherein one or more of the rings is a phenyl group. Bicyclic fused ring systems consist of a phenyl group fused to a monocyclic cycloalkenyl group, a monocyclic cycloalkyl group, or another phenyl group. The aryl groups of the present invention can be attached to the parent molecular moiety through any substitutable carbon atom in the group. Representative examples of aryl groups include, but are not limited to, indanyl, indenyl, naphthyl, phenyl, and tetrahydronaphthyl.

The term “arylalkyl,” as used herein, refers to an alkyl group substituted with one or two aryl groups.

The term “arylalkylene,” as used herein, refers to a divalent arylalkyl group, where one point of attachment to the parent molecular moiety is on the aryl portion and the other is on the alkyl portion.

The term “arylene,” as used herein, refers to a divalent aryl group.

The term “aryloxy,” as used herein, refers to an aryl group attached to the parent molecular moiety through an oxygen atom.

The term “aryloxyalkyl,” as used herein, refers to an alkyl group substituted with one or two aryloxy groups.

The term “carbonyl,” as used herein, refers to —C(O)—.

The term “cyano,” as used herein, refers to —CN.

The term “cycloalkenyl,” as used herein, refers to a non-aromatic, partially unsaturated monocyclic, bicyclic, or tricyclic ring system having three to fourteen carbon atoms and zero heteroatoms. Representative examples of cycloalkenyl groups include, but are not limited to, cyclohexenyl, octahydronaphthalenyl, and norbornylenyl.

The term “cycloalkyl,” as used herein, refers to a saturated monocyclic, bicyclic, or tricyclic hydrocarbon ring system having three to fourteen carbon atoms and zero heteroatoms. Representative examples of cycloalkyl groups include, but are not limited to, cyclopropyl, cyclopentyl, bicyclo[3.1.1]heptyl, and adamantyl.

The term “cycloalkylene,” as used herein, refers to a divalent cycloalkyl group.

The term “dialkylamino,” as used herein, refers to —NR^{x}R^{y}, wherein R^{x} and R^{y} are each alkyl groups.

The term “formyl,” as used herein, refers to —CHO.

The terms “halo,” and “halogen,” as used herein, refer to F, Cl, Br, and I.

The term “haloalkoxy,” as used herein, refers to a haloalkyl group attached to the parent molecular moiety through an oxygen atom.

The term “haloalkyl,” as used herein, refers to an alkyl group substituted by one, two, three, or four halogen atoms.

The term “heterocyclyl,” as used herein, refers to a five-, six-, or seven-membered ring containing one, two, or three heteroatoms independently selected from the group consisting of nitrogen, oxygen, and sulfur. The five-membered ring has zero to two double bonds and the six- and seven-membered rings have zero to three double bonds. The term “heterocyclyl” also includes bicyclic groups in which the heterocyclyl ring is fused to a phenyl group, a monocyclic cycloalkenyl group, a monocyclic cycloalkyl group, or another monocyclic heterocyclyl group. The heterocyclyl groups of the present invention can be attached to the parent molecular moiety through a carbon atom or a nitrogen atom in the group. Examples of heterocyclyl groups include, but are not limited to, benzothienyl, furyl, imidazolyl, indolinyl, indolyl, isothiazolyl, isoxazolyl, morpholinyl, oxazolyl, piperazinyl, piperidinyl, pyrazolyl, pyridinyl, pyrrolidinyl, pyrrolopyridinyl, pyrrolyl, thiazolyl, thienyl, and thiomorpholinyl.

The term “heterocyclylalkyl,” as used herein, refers to a heterocyclyl group attached to the parent molecular moiety through an alkyl group.

The term “heterocyclylalkylene,” as used herein, refers to a divalent heterocyclylalkyl group, where one point of attachment to the parent molecular moiety is on the heterocyclyl portion and the other is on the alkyl portion.

The term “hydroxy,” as used herein, refers to —OH.

The term “nitro,” as used herein, refers to —NO₂.

The term “oxo,” as used herein, refers to ═O.

The term “Pg,” as used herein, refers to a thiol protecting group. Exemplary thiol protecting groups include those listed in Greene and Wuts, Protective Groups in Organic Synthesis, John Wiley & Sons, New York (1991). Any thiol protecting group known in the art may be used. Examples of thiol protecting groups include, but are not limited to, acetamidomethyl, benzamidomethyl, 1-ethoxyethyl, benzoyl, and triphenylmethyl.

The term “imaging moiety,” as used herein, refers to a portion of a molecule that allows for the generation of diagnostic images. The techniques used to generate diagnostic images are known to those of ordinary skill in the art. Examples of imaging agents include, but are not limited to, radioisotopes for nuclear medicine imaging, radioisotopes for X-ray CT imaging, paramagnetic species for use in MRI imaging, echogenic entities for use in ultrasound imaging, fluorescent entities for use in fluorescence imaging, and light-active entities for use in optical imaging.

Examples of nuclear medicine imaging moieties include ¹C, ¹³N, ¹⁸F, ¹²³I, ¹²⁵I, ^{99m}Tc, ⁹⁵Tc, ¹¹¹In, ⁶²Cu, ⁶⁴Cu, ⁶⁷Ga, and ⁶⁸Ga. ¹¹C-Palmitate has been used to probe fatty acid oxidation and 11C-acetate has been used to assess oxidative metabolism in the myocardium (Circulation 1987, 76, 687-696). ¹³N-Ammonia has been used widely to image myocardial perfusion (Circulation 1989, 80, 1328-1337). Agents containing ¹⁸F have been used as imaging agents for hypoxia and cancer (Drugs of the Future 2002, 27, 655-667). The iodinated agents 15-(p-(¹²³I)-iodophenyl)-pentadecanoic acid and 15-(p-(¹²³I)-iodophenyl)-3(R,S)-methylpentadecanoic acid have been used for imaging myocardial metabolism.

Further examples of imaging moieties include X-ray absorbing or “heavy” atoms of atomic number 20 or higher. A frequently used heavy atom in X-ray imaging agents is iodine. Recently, X-ray imaging agents comprised of metal chelates and polychelates comprised of a plurality of metal ions have been disclosed (see U.S. Pat. Nos. 5,417,959 and 5,679,810). More recently, multinuclear cluster complexes have been disclosed as X-ray imaging agents (see U.S. Pat. No. 5,804,161; WO91/14460; and WO92/17215). Representative metals include Re, Sm, Ho, Lu, Pm, Y, Bi, Pd, Gd, La, Au, Yb, Dy, Cu, Rh, Ag, and Ir.

MRI imaging agents may contain one or more paramagnetic metal ions. The ions may be present in the form of metal chelates, complexes, or metal oxide particles. Examples of chelators for paramagnetic metal ions in MRI imaging agents are described in U.S. Pat. Nos. 5,412,148 and 5,760,191. Examples of polychelants useful for complexing more than one paramagnetic metal ion are found in U.S. Pat. Nos. 5,801,228; 5,567,411; and 5,281,704. U.S. Pat. No. 5,520,904 describes particulate compositions comprised of paramagnetic metal ions for use as MRI imaging agents. Examples of metals include Gd³⁺, Fe³⁺, In³⁺, and M²⁺.

The ultrasound imaging agents may comprise a microbubble of a biocompatible gas, a liquid carrier, and a sufactant microsphere. As used herein, the term “liquid carrier” means aqueous solution and the term “surfactant” means any amphiphilic material which may produce a reduction in interfacial tension in a solution. A list of suitable surfactants for forming surfactant microspheres is disclosed, for example, in EP0727225A2. The term “surfactant microsphere” includes microspheres, nanospheres, liposomes, vesicles, and the like. The biocompatible gas can be any physiologically accepted gas, including, for example, air or a fluorocarbon, such as a C₃-C₅ perfluoroalkane, which provides the difference in echogenicity and thus the contrast in ultrasound imaging. The gas may be encapsulated, contained, or otherwise constrained in or by the microsphere to which is attached the remainder of the molecule. The attachment can be covalent, ionic, or by van der Waals forces. Specific examples of such contrast agents include, for example, lipid encapsulated perfluorocarbons with a plurality of tumor neovasculature receptor binding peptides, polypeptides, or peptidomimetics. Examples of gas filled imaging moieties include those found in U.S. patent application Ser. No. 09/931,317, filed Aug. 16, 2001, and U.S. Pat. Nos. 5,088,499; 5,547,656; 5,228,446; 5,585,112; and 5,846,517.

The term “microbubbles,” as used herein, refer to vesicles which are generally characterized by the presence of one or more membranes or walls surrounding an internal void that is filled with a gas or precursor thereto. Exemplary microbubbles include, for example, liposomes, micelles, and the like.

“Ancillary” or “co-ligands” are ligands that may be incorporated into a radiopharmaceutical during its synthesis. They may serve to complete the coordination sphere of the radionuclide together with the chelator or radionuclide bonding unit of the reagent. For radiopharmaceuticals comprised of a binary ligand system, the radionuclide coordination sphere may be composed of one or more chelators or bonding units from one or more reagents and one or more ancillary or co-ligands, provided that there are a total of two types of ligands, chelators, or bonding units. For example, a radiopharmaceutical comprised of one chelator or bonding unit from one reagent and two of the same ancillary or co-ligands and a radiopharmaceutical comprised of two chelators or bonding units from one or two reagents and one ancillary or co-ligand are both considered to be comprised of binary ligand systems. For radiopharmaceuticals comprised of a ternary ligand system, the radionuclide coordination sphere may be composed of one or more chelators or bonding units from one or more reagents and one or more of two different types of ancillary or co-ligands, provided that there are a total of three types of ligands, chelators, or bonding units. For example, a radiopharmaceutical comprised of one chelator or bonding unit from one reagent and two different ancillary or co-ligands is considered to be comprised of a ternary ligand system. Ancillary or co-ligands useful in the preparation of radiopharmaceuticals and in diagnostic kits useful for the preparation of said radiopharmaceuticals may be comprised of one or more oxygen, nitrogen, carbon, sulfur, phosphorus, arsenic, selenium, and tellurium donor atoms. A ligand can e a transfer ligand in the synthesis of a radiopharmaceutical and also serve as an ancillary or co-ligand in another radiopharmaceutical. Whether a ligand is termed a transfer or ancillary or co-ligand depends on whether the ligand remains in the radionuclide coordination sphere in the radiopharmaceutical, which is determined by the coordination chemistry of the radionuclide and the chelator or bonding unit of the reagent or reagents.

The terms “chelator” and “bonding unit,” as used herein, refer to a group on a reagent that binds to a metal ion through the formation of chemical bonds with one or more donor atoms. Examples of chelators are described in U.S. Pat. No. 6,511,648. In one example of the present disclosure the bonding unit is selected from 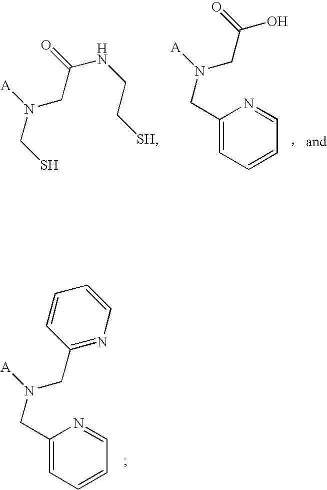
wherein A is a bond to the compound of formula (I). The synthesis of these compounds is disclosed in WO03/086476.

In another example of the present disclosure the bonding unit is selected from 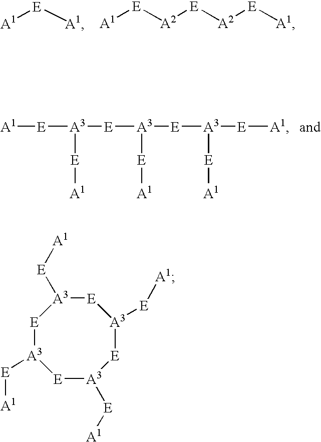
wherein

each A¹ is independently selected from a bond to the compound of formula (I), —NR⁵R⁶, —SH, —S(Pg), —OH, —PR⁵R⁶, and —P(O)R⁷R⁸;

each A² is independently selected from S, O, and PR⁵;

A³ is N;

each E is independently selected from C₁₋₁₀alkylene substituted with 0-3 R⁹ groups, C₆₋₁₀arylene substituted with 0-3 R⁹ groups, C₃₋₁₀cycloalkylene substituted with 0-3 R⁹ groups, heterocyclyl-C₁₋₁₀alkylene substituted with 0-3 R⁹ groups, C₆₋₁₀aryl-C₁₋₁₀alkylene substituted with 0-3 R⁹ groups, and heterocyclyl substituted with 0-3 R⁹ groups;

R⁵ and R⁶ are each independently selected from a bond to the compound of formula (I), C₁₋₁₀alkyl substituted with 0-3 R⁸ groups, C₆₋₁₀aryl substituted with 0-3 R⁸ groups, C₃₋₁₀cycloalkyl substituted with 0-3 R⁸ groups, heterocyclyl-C₁₋₁₀alkyl substituted with 0-3 R⁸ groups, and heterocyclyl substituted with 0-3 R⁸ groups;

R⁷ and R⁸ are each independently selected from a bond to the compound of formula (I), C₁₋₁₀alkyl substituted with 0-3 R⁹ groups, C₆₋₁₀aryl substituted with 0-3 R⁹ groups, C₃₋₁₀cycloalkyl substituted with 0-3 R⁹ groups, heterocyclyl-C₁₋₁₀alkyl substituted with 0-3 R⁹ groups, C₆₋₁₀aryl-C₁₋₁₀alkyl substituted with 0-3 R⁹ groups, heterocyclyl substituted with 0-3 R⁹ groups, and hydroxy;

each R⁹ is independently selected from a bond to the compound of formula (I), C₂₋₄alkenyl, C₁₋₆alkoxy, C₁₋₆alkoxycarbonyl, di(C₁₋₆alkyl)amino, C₁₋₆alkylcarbonyl, amino, cyano, C₃₋₆cycloalkyl, formyl, halo, haloalkoxy, haloalkyl, hydroxy, nitro, and oxo; and

Pg is a thiol protecting group;

provided at least one bond to the compound of formula (I) is present. These compounds as well as their synthesis are known to those of ordinary skill in the art.

The term “pharmaceutically acceptable salt,” as used herein, refers to any pharmaceutically acceptable salt of a compound of the disclosure that, upon administration to a recipient, is capable of providing (directly or indirectly) a compound of this disclosure or a metabolite or residue thereof. Typically, derivatives are those that increase the bioavailability of the compounds of the disclosure when such compounds are administered to a mammal (e.g., by allowing an orally administered compound to be more readily absorbed into the blood) or which enhance delivery of the parent compound to a biological compartment (e.g., the brain or lymphatic system) relative to the parent species.

As used herein, the phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problem or complication, commensurate with a reasonable benefit/risk ratio.

The compounds of the present disclosure may be used in a method of imaging including methods of imaging in a matient comprising administering one or more compounds to the patient by injection, infusion, or any other known method, and imaging the area of the patient wherein the event of interest is located.

The dosage to be administered and the particular mode of administration will vary depending upon such factors as age, weight, and particular region to be treated, as well as the particular agent used, the diagnostic use contemplated, and the form of the formulation (e.g., suspension, emulsion, microsphere, liposome, or the like), as is known by those of ordinary skill in the art.

Typically, the dosage is administered at lower levels and is increased until the desirable diagnostic effect is achieved. In one embodiment, the compounds of the invention may be administered by intravenous injection, usually in saline solution, at a dose of about 0.1 to about 100 mCi per 70 kg body weight (and all combinations and subcombinations of dosage ranges and specific dosages therein), or, for example, at a dose of about 0.5 to about 50 mCi. Imaging is performed using techniques known to those of ordinary skill in the art.

For use as nuclear medicine imaging agents, the compositions of the present disclosure, administered by intravenous injection, will typically range in dose from about 0.5 μmol/kg to about 1.5 mmol/kg (and all combinations and subcombinations of dosage ranges and specific dosages therein), for example about 0.8 μmol/kg to about 1.2 mmol/kg.

For use as MRI imaging agents, the compositions of the present disclosure may be used in a similar manner as other MRI agents as described in U.S. Pat. Nos. 5,155,215 and 5,087,440; Magn. Reson. Med. 1986, 3, 808; Radiology 1988, 166, 835; and Radiology 1988, 166, 693. Generally, sterile aqueous solutions of the contrast agents may be administered to a patient intravenously in dosages ranging from about 0.01 to about 1.0 mmoles per kg body weight (and all combinations and subcombinations of dosage ranges and specific dosages therein).

The ultrasound imaging agents of the present disclosure may be administered by intravenous injection in an amount from about 10 to about 30 μL (and all combinations and subcombinations of dosage ranges and specific dosages therein) of the echogenic gas per kg body weight or by infusion at a rate of approximately 3 μL/kg/min.

The compounds of the present disclosure may contain one or more chiral centers and exist in different optically active forms. It should be understood that the present disclosure encompasses all stereochemical isomeric forms, or mixtures thereof. When compounds of formula (I) contain one chiral center, the compounds exist in two enantiomeric forms. The enantiomers may be resolved by methods known to those skilled in the art, for example, by formation of diastereoisomeric salts which may be separated by crystallization, gas-liquid, or liquid chromatography; or by selective reaction of one enantiomer with an enantiomer-specific reagent. It will be appreciated that where the desired enantiomer is converted into another chemical entity by a separation technique, then an additional step is required to form the desired enantiomeric form. Alternatively, specific enantiomers may be synthesized by asymmetric synthesis using optically active reagents, substrates, catalysts, or solvents, or by converting one enantiomer into the other by asymmetric transformation. Starting compounds of particular stereochemistry are either commercially available or can be made and resolved by techniques known in the art.

When any variable occurs more than one time in any substituent or in any formula, its definition in each occurrence is independent of its definition at every other occurrence. Thus, for example, if a group, or plurality of groups, is shown to be substituted with up to two R¹⁰, then said group(s) may be optionally substituted with up to two R¹⁰, and R¹⁰ at each occurrence in each group is selected independently from the defined list of R¹⁰. Also, by way of example, for the group —N(R¹¹)₂, each of the two R¹¹ substituents on N is independently selected from the defined list of possible R¹¹. Combinations of substituents and/or variables are permissible only if such combinations result in stable compounds.

The present disclosure will now be described in connection with certain embodiments which are not intended to limit its scope. On the contrary, the present disclosure covers all alternatives, modifications, and equivalents as can be included within the scope of the claims. Thus, the following examples will illustrate one practice of the disclosure, it being understood that the examples are for the purpose of illustration and are presented to provide what is believed to be the most useful and readily understood description of its procedures and conceptual aspects.

Abbreviations used within the examples are as follows: PPTS for pyridinium p-toluenesulfonate; DCM for dichloromethane; THP for tetrahydrpyran; nBuLi for n-butyllithium; THF for tetrahydrofuran; TsOH for p-toluenesulfonic acid; MeOH for methanol; Hr for hour; TsCl for p-toluenesulfonyl chloride; DMAP for N,N-dimethylaminopyridine; DIEA for N,N-diisopropylethylamine; EtOH for ethanol; Ts for p-toluenesulfonyl; AcN for acetonitrile; TBSCl for tert-butyldimethylsilyl chloride; DMF for N,N-dimethylformamide; TBS for tert-butyldimethylsilyl; Concd. for concentrated; TBAF for tetrabutylammonium fluoride; Bu for butyl; NaOEt for sodium ethoxide; Me for methyl; Et₂O for diethyl ether; and Bu₄NF for tetrabutylammonium fluoride.

### EXAMPLE 1

### Synthesis of 2,3-dimethoxy-4-hydroxy-5-methyl-6-(9-[¹⁸F]fluorononyl)pyridine

### Synthesis of 9-tetrahydropyranylox-1-bromononane

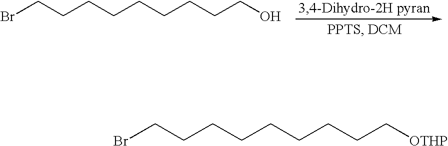

9-Bromo-1-nonanol (9 mmol) is dissolved in methylene chloride (10 mL) and to it is added pyridinium p-toluenesulfonate (0.009 mmol) and dihydropyran (13.5 mmol). The mixture is stirred for 4 hours after which the solution is poured in a separatory funnel and washed with water and brine and dried over MgSO₄. The solution is filtered and concentrated in vacuo and purified using a short plug of silica to provide the purified product.

### Synthesis of 2,3-dimethoxy-4-benzyloxy-5-methyl-6-(9-(2-tetrahydropyranoxynonyl)-pyridine

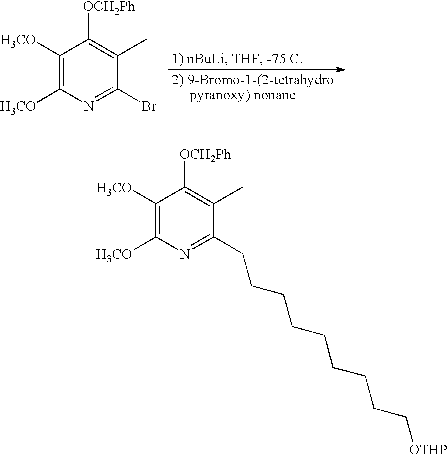

To 2,3-dimethoxy-4-benzyloxy-5-methyl-6-bromopyridine (prepared according to the procedure described in J. Am Chem. Soc. 1977, 99, 7014-7019; 0.74 mmol) placed in a round bottom flask is added 8 mL anhydrous tetrahydrofuran under nitrogen and the solution is cooled to −75° C. n-Butyllithium solution (2.5M in hexane, 0.81 mmol) is then added to the above mixture and the mixture is stirred for 15 minutes. 9-Tetrahydropyranyloxy-1-bromononane (1.11 mmol) is then added by syringe and the mixture is stirred for 3 hours at −75° C. Water (1 mL) is then added to the above mixture and the mixture is stirred for 5 minutes after which it is poured into a separatory funnel and extracted with methylene chloride. The extracts are filtered through a pad of diatomaceous earth (such as Celite®), washed with brine, dried over MgSO₄, filtered, and concentrated. The concentrate is purified by silica gel chromatography to obtain 2,3-dimethoxy-4-benzyloxy-5-methyl-6-(9-tetrahydropyranoxynonyl)pyridine as the purified product.

### Synthesis of 2,3-dimethoxy-4-benzyloxy-5-methyl-6-(9-hydroxynonyl)pyridine

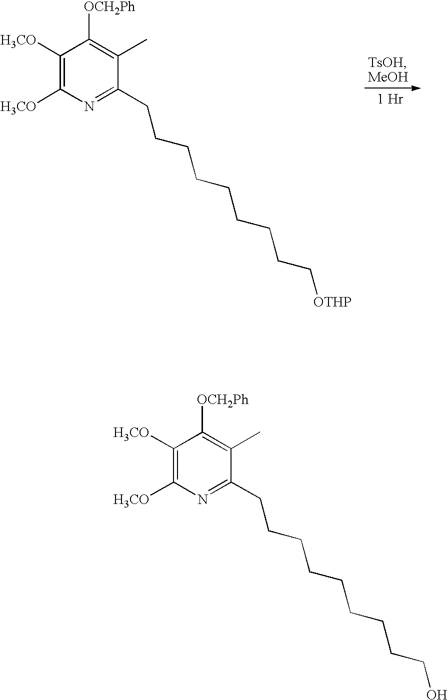

2,3-Dimethoxy-4-benzyloxy-5-methyl-6-(9-tetrahydropyranoxynonyl)pyridine (1.03 mmol) is dissolved in methanol and to it is added p-toluenesulfonic acid (0.1 mmol). The reaction mixture is stirred for 1 hour after which it is washed with water and brine, dried over MgSO₄, filtered, and concentrated. The concentrate is purified by silica gel chromatography to obtain 2,3-dimethoxy-4-benzyloxy-5-methyl-6-(9-hydroxynonyl)-pyridine as the purified product.

### Synthesis of 2,3-dimethoxy-4-benzyloxy-5-methyl-6-(9-tosyloxynonyl)pyridine

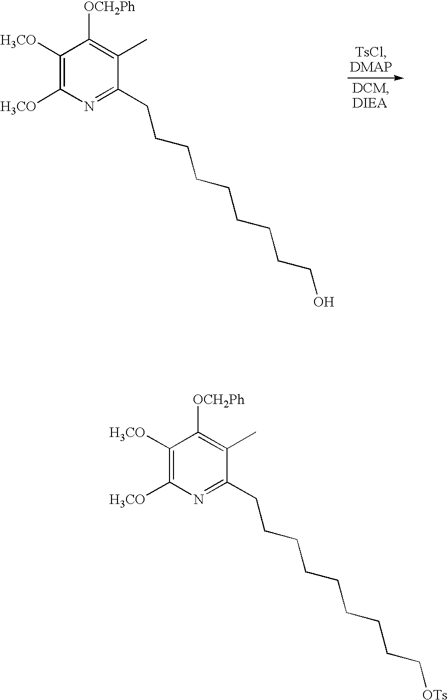

2,3-Dimethoxy-4-benzyloxy-5-methyl-6-(9-hydroxynonyl)pyridine (0.49 mmol) is charged to a round bottom flask and to it is added 5 mL methylene chloride. N,N-Dimethylaminopyridine (0.58 mmol), p-toluenesulfonyl chloride (0.58 mmol), and diisopropylethylamine (2.45 mmol, 0.43 mL) are then added to the flask and the reaction mixture is stirred for 4 hours. The mixture is then poured into a separatory funnel and water is added. The layers are separated and the organic layer is washed with brine, dried over MgSO₄, filtered, and concentrated. The concentrate is purified by silica gel chromatography to obtain 2,3-dimethoxy-4-benzyloxy-5-methyl-6-(9-tosyloxynonyl)-pyridine as the purified product.

### Synthesis of 2,3-dimethoxy-4-hydroxy-5-methyl-6-(9-tosyloxynonyl)pyridine

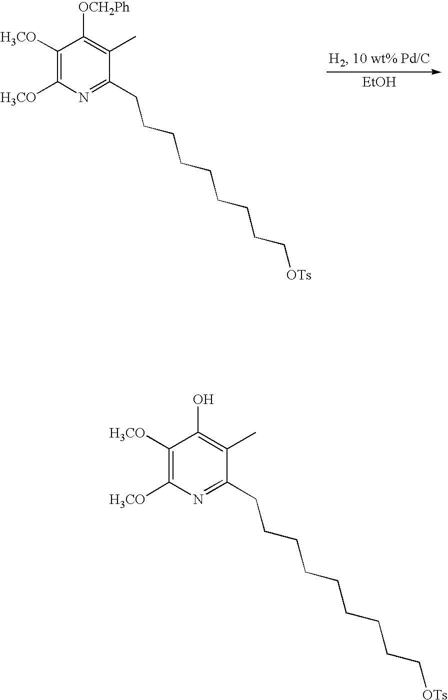

To a pre-equilibrated mixture of 10 wt % Pd/C (0.11 g) in 2 mL ethanol is added 2,3-dimethoxy-4-benzyloxy-5-methyl-6-(9-tosyloxynonyl)pyridine (0.36 mmol). The reaction mixture is hydrogenated at room temperature and atmospheric pressure and stirred until the absorption of hydrogen ceases. The solution is then filtered through a pad of diatomaceous earth (such as Celite®) and the pad is washed with ethanol (5 mL). The ethanol is then evaporated in vacuo to obtain 2,3-dimethoxy-4-hydroxy-5-methyl-6-(9-tosyloxynonyl)pyridine as the purified product.

### Synthesis of Z 3-dimethoxy-4-hydroxy-5-methyl-6-(9-fluorononyl)pyridine

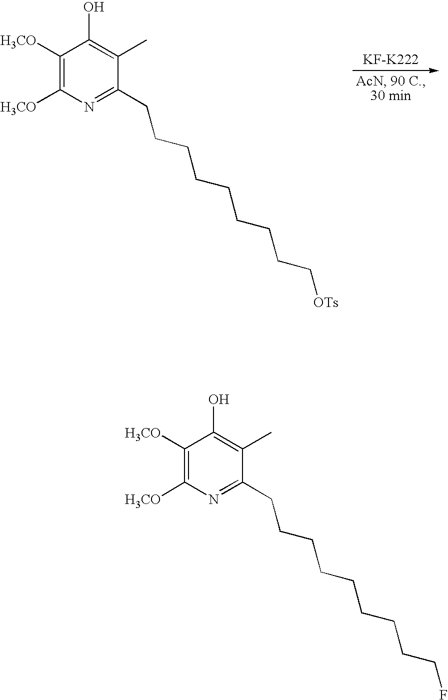

A 15 mL round bottom flask is charged with potassium fluoride (0.1 mmol) and Kryptofix 222 (0.1 mmol). Acetonitrile (2 mL) is then added and the reaction mixture is stirred until the solution turns clear. 2,3-Dimethoxy-4-hydroxy-5-methyl-6-(9-tosyloxynonyl)pyridine (0.1 mmol) dissolved in 1 mL acetonitrile is then added to the above mixture. The round bottom flask is then fitted with a reflux condenser and immersed in a oil bath at 90° C. The mixture is refluxed for 30 minutes after which it is cooled and the acetonitrile evaporated in vacuo. The crude residue obtained is purified by silica gel chromatography to obtain 2,3-dimethoxy-4-hydroxy-5-methyl-6-(9-fluorononyl)pyridine as the purified product.

### Synthesis of 2,3-dimethoxy-4-hydroxy-5-methyl-6-(9-[¹⁸F]-fluorononyl)pyridine

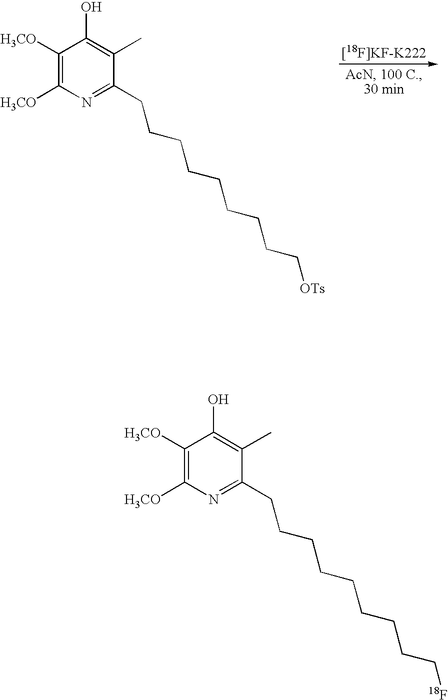

To a 5 mL reaction vial containing 500 mCi of ¹⁸F in 350 mg of ¹⁸O water is added a 1 mL solution consisting of 10 mg of Kryptofix-222, 1 mg potassium carbonate, 0.005 mL water and 0.95 mL acetonitrile. The vial is heated to remove all the solvents and dry acetonitrile (1 mL) is added to the vial. This is also removed by evaporation. 2,3-Dimethoxy-4-hydroxy-5-methyl-6-(9-tosyloxynonyl)pyridine (5 mg) in acetonitrile is then added to it. The vial is sealed and heated for 30 minutes at 100° C. The mixture is diluted with dichloromethane and passed through a chromatography cartridge and eluted with tetrahydrofuran. The solvent is evaporated to provide the desired product.

### EXAMPLE 2

### Synthesis of 2,3-dimethoxy-4-hydroxy-5-methyl-6-(2-(4-[¹⁸F]fluorophenyl)ethyl)pyridine

### Synthesis of 2,3-dimethoxy-4-tert-butyldimethylsilyloxy-5-methyl-6-bromopyridine

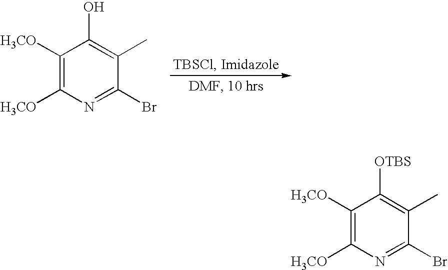

2,3-Dimethoxy-4-hydroxy-5-methyl-6-bromopyridine (prepared according to the procedure described in J. Am Chem. Soc. 1977, 99, 7014-7019, 2.02 mmol) is placed in a round bottom flask and to it is added 5 mL DMF. Tert-butyldimethylsilyl chloride (3.03 mmol) and imidazole (5.05 mmol) are then added to the above mixture and the mixture is stirred for 10 hours. The DMF is then removed in vacuo and the resulting residue is taken up in methylene chloride and washed with water. The organic layer is then washed with brine, dried over MgSO₄, and filtered. The solution is concentrated and the crude mixture is purified by silica gel chromatography to obtain the desired compound as the purified product.

### Synthesis of 2,3-dimethoxy-4-tert-butyldimethylsilyloxy-5-methyl-6-(2-(4-nitrophenyl)ethyl)pyridine

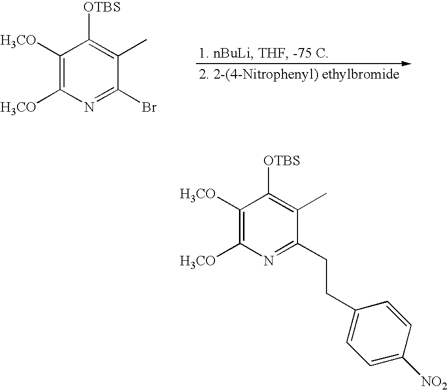

A round bottom flask is charged with 2,3-dimethoxy-4-tert-butyldimethylsilyloxy-5-methyl-6-bromopyridine (0.83 mmol) and to it is added 8 mL anhydrous tetrahydrofuran under nitrogen. The solution is cooled to −75° C. n-Butyllithium solution (2.5M in hexane, 0.91 mmol) is then added to the above mixture and the mixture is stirred for 15 minutes. 2-(4-Nitrophenyl)ethylbromide (1.24 mmol) is then added by syringe and the mixture is stirred for 3 hours at −75° C. Water (2 mL) is then added to the above mixture and the mixture is stirred for 5 minutes after which it is poured into a separatory funnel and extracted with methylene chloride. The solution is filtered through a pad of diatomaeous earth (Celite®), washed with brine, dried over MgSO₄, filtered, and concentrated. The crude product is purified by silica gel chromatography to obtain 2,3-dimethoxy-4-tert-butyldimethylsilyloxy-5-methyl-6-(2-(4-nitrophenyl)ethyl)pyridine as the purified product.

### Synthesis of 2,3-dimethoxy-4-hydroxy-5-methyl-6-(2-(4-nitrophenyl)ethyl)pyridine

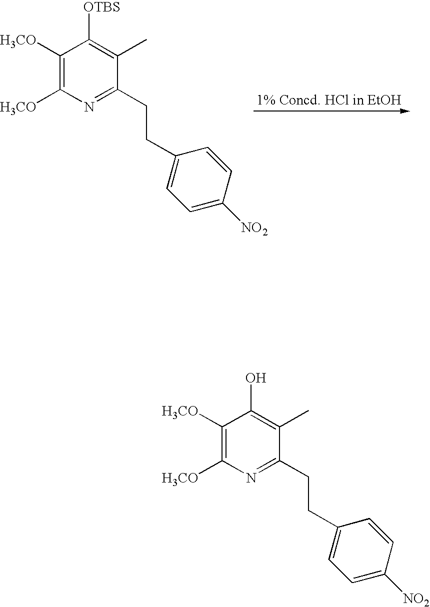

To a round bottom flask charged with 2,3-dimethoxy-4-tert-butyldimethylsilyloxy-5-methyl-6-(2-(4-nitrophenyl)ethyl)pyridine (0.578 mmol) in added 10 mL of 1% conc. HCl in ethanol solution. The above solution is then stirred for 30 minutes after which it is poured into a separatory funnel and extracted with methylene chloride. The organic layer is then washed with water and then with brine. It is then dried over MgSO₄ and filtered. The solvent is then removed in vacuo and the crude product obtained is purified using a short plug of silica gel using to obtain 2,3-dimethoxy-4-hydroxy-5-methyl-6-(2-(4-nitrophenyl)ethyl)pyridine as the purified product.

### Synthesis of 2,3-dimethoxy-4-hydroxy-5-methyl-6-(2-(4-fluorophenyl)ethyl)pyridine

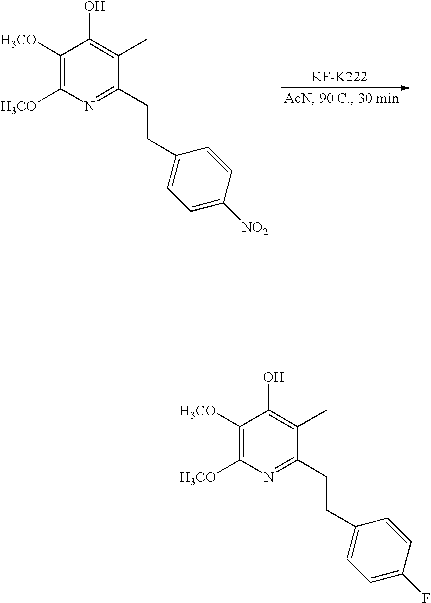

A 15 mL round bottom flask is charged with potassium fluoride (0.15 mmol) and Kryptofix 222 (0.15 mmol). 2 mL Acetonitrile is then added and the reaction mixture is stirred until the solution turns clear. 2,3-Dimethoxy-4-hydroxy-5-methyl-6-(2-(4-nitrophenyl)ethyl)pyridine (0.15 mmol) dissolved in 1 mL acetonitrile is then added to the above mixture. The round bottom flask is then fitted with a reflux condenser and immersed in a oil bath at 90° C. The mixture is refluxed for 30 minutes after which it is cooled and the acetonitrile evaporated in vacuo. The crude residue obtained is purified by silica gel chromatography to obtain 2,3-dimethoxy-4-hydroxy-5-methyl-6-(2-(4-fluorophenyl)ethyl)pyridine as the purified product.

### Synthesis of 2,3-dimethoxy-4-hydroxy-5-methyl-6-(2-(4-[¹⁸F]-fluorophenyl)ethyl)pyridine

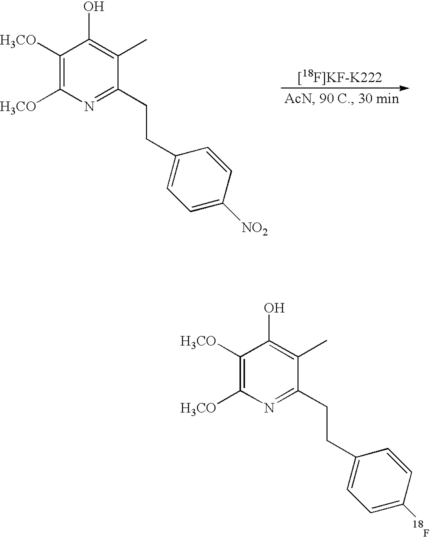

To a 5 mL reaction vial containing 500 mCi of ¹⁸F in 350 mg of ¹⁸O water is added a 1 mL solution consisting of 10 mg of Kryptofix, 1 mg potassium carbonate, 0.005 mL water and 0.95 mL acetonitrile. The vial is heated to remove all the solvents and dry acetonitrile (1 mL) is added to the vial. This is also removed by evaporation. 2,3-Dimethoxy-4-hydroxy-5-methyl-6-(2-(4-nitrophenyl)ethyl)pyridine (5 mg) in acetonitrile is then added to it. The vial is sealed and heated for 30 minutes at 100° C. The mixture is diluted with dichloromethane and passed through a chromatography cartridge and eluted with tetrahydrofuran. The solvent is evaporated to provide the desired product.

### EXAMPLE 3

### Synthesis of 2,3-dimethoxy-4-hydroxy-5-methyl-6-(2-(4-(2-[¹⁸F]-fluoroethoxy)phenyl)ethyl)pyridine

### Synthesis of 4-Bromoacety phenol

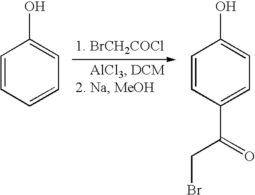

Phenol (21.1 mmol) is dissolved in 20 mL of anhydrous methylene chloride in a round bottom flask and the mixture is cooled to 0° C. using an ice bath. Aluminum chloride (63.8 mmol) is added to the above solution in one portion and the reaction is stirred under nitrogen for 3 hours. Water is then added very slowly to the reaction until all excess aluminum chloride is consumed and the mixture is then poured into a separatory funnel and extracted with diethyl ether. The ether layer is then washed with water and brine and dried over MgSO₄ and filtered. The solvent is then evaporated in vacuo and the crude oil obtained is then dissolved in methanol. Sodium (100 mg) is then added to the above solution and this is stirred for 3 hours. Water is slowly added to the above mixture and this is then extracted with diethyl ether. The ether layer is washed with water and brine and dried over MgSO₄ and filtered. The crude residue obtained after removing the methanol is subjected to purification using a short plug of silica gel to obtain 4-bromoacetylphenol as the purified product.

### Synthesis of 4-(2-bromoethyl)phenol

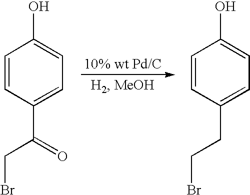

To a round bottom flask charged with 4-bromoacetylphenol (4.6 mmol) is added 15 mL methanol. 10 wt % Pd/C (10 wt %, 0.10 g) is then added to it and the mixture is hydrogenated using a balloon filled with hydrogen. The reaction allowed to sit for 10 hours at which time it's filtered through a pad of diatomaceous earth (Celite®). The filtrate is then concentrated in vacuo to obtain 4-(2-bromoethyl)phenol as the purified product.

### Synthesis of 4-(2-bromoethyl) tert-butyldimethylsilyloxy benzene

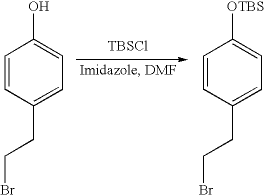

4-(2-Bromoethyl)phenol (3.7 mmol) is dissolved in DMF in a round bottom flask. Imidazole (9.25 mmol) and tert-butyldimethylsilyl chloride are then added to the above mixture and the reaction stirred for 10 hours. The DMF is removed in vacuo and the crude mixture obtained is dissolved in methylene chloride. This is then washed with water and brine and dried over MgSO₄ and filtered. The crude product obtained after removing the organic solvent is purified using a short plug of silica to obtain 4-(2-bromoethyl) tert-butyldimethylsilyloxy benzene as the purified product.

### Synthesis of 2,3-dimethoxy-4-benzyloxy-5-methyl-6-(2-(4-tert-butyldimethylsilyloxyphenyl)ethyl)pyridine

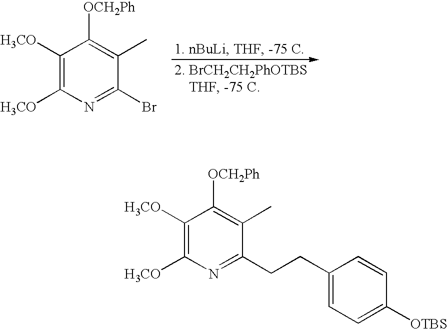

In a round bottom flask is placed 2,3-dimethoxy-4-benzyloxy-5-methyl-6-bromopyridine (0.74 mmol) and to this is added anhydrous THF (10 mL). n-Butyllithium (2.5 M in hexanes, 0.815 mmol) is then added by syringe and the reaction is stirred for 15 minutes at −75° C. 4-(2-Bromoethyl) tert-butyldimethylsilyloxybenzene (1.11 mmol) is then added to the above mixture and the reaction is stirred for 3 hours. Water (1 mL) is then added to the above mixture and the mixture is stirred for 5 minutes after which it is poured into a separatory funnel and extracted using methylene chloride. The solution is filtered through a pad of diatomaceous earth (Celite®), washed with brine, dried over MgSO₄, and filtered. The crude product obtained after removing the solvent is purified by silica gel chromatography to obtain 2,3-dimethoxy-4-benzyloxy-5-methyl-6-(2-(4-tert-butyldimethylsilyloxyphenyl)ethyl)pyridine as the purified product.

### Synthesis of 2,3-dimethoxy-4-benzyloxy-5-methyl-6-(2-(4-hydroxyphenyl)ethyl)pyridine

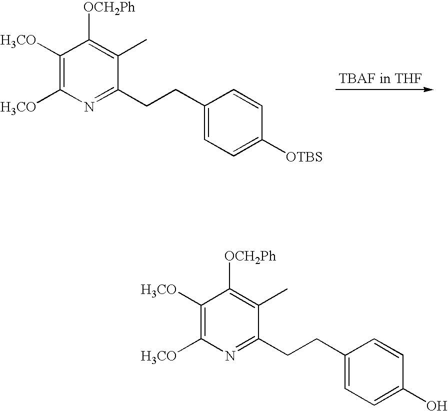

To 2,3-dimethoxy-4-benzyloxy-5-methyl-6-(2-(4-tert-butyldimethylsilyloxy phenyl)ethyl)pyridine (0.405 mmol) in a round bottom flask is added tetrabutylammonium fluoride solution (1.216 mmol, 1M in THF). The mixture is stirred for 2 hours after which all the solvent is removed in vacuo and the crude product is purified using a short plug of silica to obtain 2,3-dimethoxy-4-benzyloxy-5-methyl-6-(2-(4-hydroxyphenyl)ethyl)pyridine as the purified product.

### Synthesis of 2,3-dimethoxy-4-benzyloxy-5-methyl-6-(2-(4-(2-fluoroethoxy)phenyl)ethyl)-pyridine

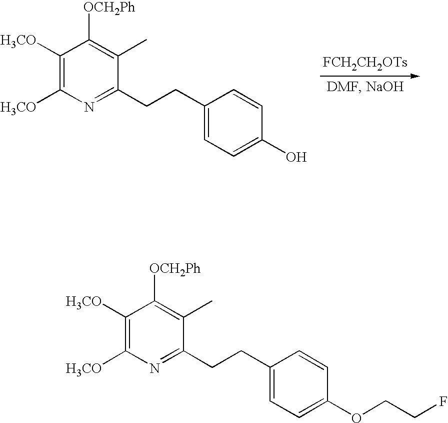

A round bottom flask is charged with 2,3-dimethoxy-4-benzyloxy-5-methyl-6-(2-(4-hydroxyphenyl)ethyl)pyridine (0.131 mmol) and to it is added 2 mL of anhydrous DMF. This is followed by the addition of sodium hydroxide solution (0.131 mmol of NaOH) and fluoroethyl tosylate (0.196 mmol). The reaction mixture is then immersed in an oil bath preheated to 90° C. and the reaction is stirred for 30 minutes. The mixture is then cooled to room temperature and the DMF is removed in vacuo. The residue obtained is dissolved in ethyl acetate and washed with water and brine, dried over MgSO₄, and filtered. The crude residue obtained after concentrating the organic solvent is purified by silica gel chromatography to obtain 2,3-dimethoxy-4-benzyloxy-5-methyl-6-(2-(4-(2-fluoroethoxy)phenyl)ethyl)pyridine as the purified product.

### Synthesis of 2,3-dimethoxy-4-hydroxy-5-methyl-6-(2-(4-(2-fluoroethyloxy)phenyl)ethyl)-pyridine

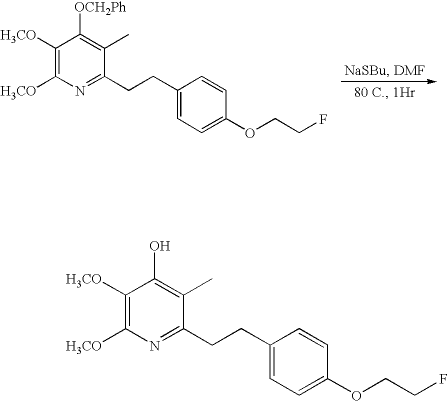

To 2,3-dimethoxy-4-benzyloxy-5-methyl-6-(2-(4-(2-fluoroethyloxy)phenyl)ethyl)pyridine (0.0705 mmol) placed in a round bottom flask is added 0.2 mL DMF. Sodium butylmercaptide (0.141 mmol) in DMF is then added to the mixture and this is kept at 80° C. for 1 hour. A few drops of saturated NH₄Cl solution followed by 50 μL of water are then added to the above mixture. The resulting mixture is then extracted with diethyl ether, washed with water and brine and dried over MgSO₄. The crude is purified using silica gel chromatography to obtain 2,3-dimethoxy-4-hydroxy-5-methyl-6-(2-(4-(2-fluoroethyloxy)phenyl)ethyl)pyridine as the purified product.

### Synthesis of 2,3-dimethoxy-4-hydroxy-5-methyl-6-(2-(4-(2-[¹⁸F]-fluoroethoxy)phenyl)ethyl)pyridine

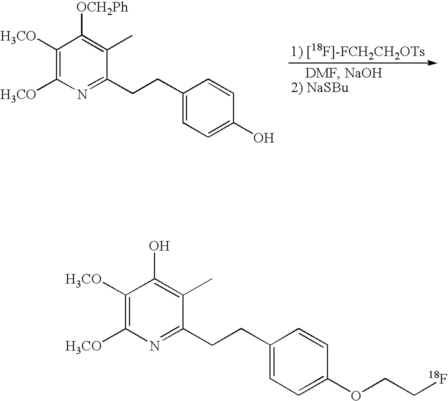

To a 3 mL conical reaction vial charged with 2,3-dimethoxy-4-benzyloxy-5-methyl-6-(2-(4-hydroxyphenyl)ethyl)pyridine (5 mg, 0.0131 mmol) is added 0.3 mL of anhydrous DMF. This is followed by the addition of sodium hydroxide solution (5N, 2.9 μL, 0.0145 mmol). The solution is heated to 90° C. for 5 minutes after which a solution of 2-[¹⁸F]fluoroethyltosylate (prepared according to the procedure described in J. Labelled Compds. Radiopharm. 2001, 44, 627-642; 300-600 MBq) in DMF (300 μL) is added and the resulting mixture is stirred for 5 minutes at 90° C. A solution of sodium butylmercaptide in DMF (0.0131 mmol) is then added to the above mixture and this is stirred for 10 minutes. The solution is then loaded on to a HPLC column followed by a solid phase column to elute the product in methanol.

### EXAMPLE 4

### Synthesis of 2-(2-(4-[¹⁸F]-fluorophenyl)ethyl)-3-methyl-4-hydroxyquinoline

### Synthesis of 2,3-dimethyl-4-tert-butyldimethylsilyloxyquinoline

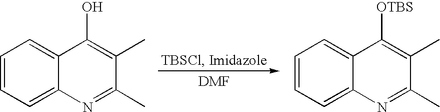

To a solution of 2,3-dimethyl-4-hydroxyquinoline (prepared according to the procedure described in J. Chem. Soc. 1939, 563-565; 5.77 mmol) in 8 mL DMF is added tert-butyldimethylsilyl chloride (8.66 mmol) and imidazole (14.4 mmol). The solution is stirred overnight after which all DMF is removed in vacuo and the resulting residue is dissolved in diethyl ether, washed with water and brine, dried over MgSO₄ and filtered. Purification using a short plug of silica gel provides pure 2,3-dimethyl-4-tert-butyldimethylsilyloxy quinoline.

### Synthesis of 2-(2-(4-nitrophenyl)ethyl)-3-methyl-4-tert-butyldimethylsilyloxyquinoline

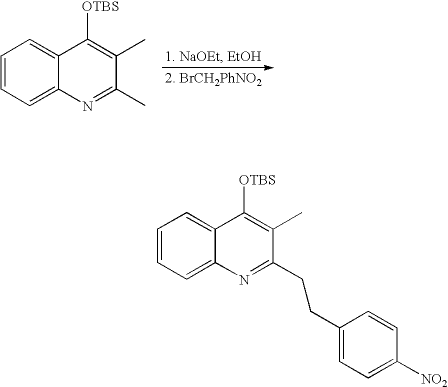

2,3-Dimethyl-4-tert-butyldimethylsilyloxyquinoline (1.741 mmol) is dissolved in anhydrous ethanol (5 mL) and to it is added sodium ethoxide (2.089 mmol). The above mixture is stirred for 10 minutes after which 4-nitro benzylbromide (2.611 mmol) is added. The reaction mixture is stirred for 5 hours after which water is slowly added to the reaction mixture. The contents of the flask are then poured into a separatory funnel and the solution extracted with methylene chloride. The organic layer is then washed with water and brine, dried over MgSO₄, filtered, and concentrated. Purification using silica gel chromatography provides pure 2-(2-(4-nitrophenyl)ethyl)-3-methyl-4-tert-butyldimethylsilyloxyquinoline.

### Synthesis of 2-(2-(4-nitrophenyl)ethyl)-3-methyl-4-hydroxyquinoline

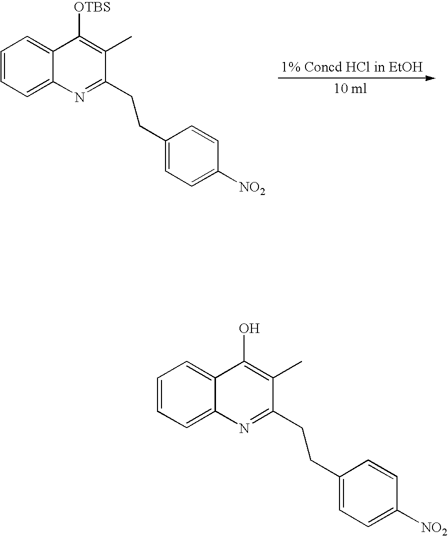

To a round bottom flask charged with 2-(2-(4-nitrophenyl)ethyl)-3-methyl-4-tert-butyldimethylsilyloxyquinoline (1.184 mmol) is added 10 mL of 1% conc. HCl in ethanol solution. The above solution is then stirred for 30 minutes after which it is poured into a separatory funnel and extracted with methylene chloride. The organic layer is then washed with water and then with brine. It is then dried over MgSO₄ and filtered. The solvent is then removed in vacuo and the crude product obtained is purified using a short plug of silica gel to obtain 2-(2-(4-nitrophenyl)ethyl)-3-methyl-4-hydroxyquinoline as the purified product.

### Synthesis of 2-(2-(4-fluorophenyl)ethyl)-3-methyl-4-hydroxyquinoline

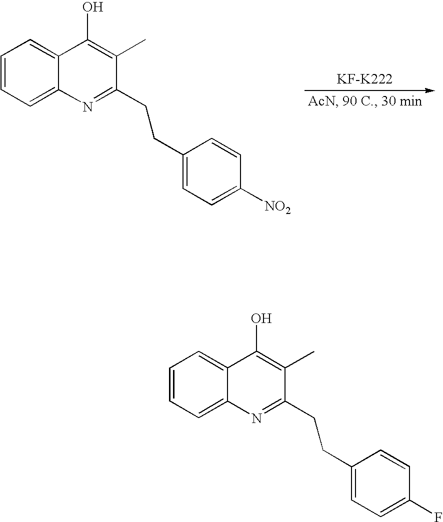

A 15 mL round bottom flask is charged with potassium fluoride (0.324 mmol) and Kryptofix 222 (0.324 mmol). 3 mL Acetonitrile is then added and the reaction mixture is stirred until the solution turns clear. 2-(2-(4-Nitrophenyl)ethyl)-3-methyl-4-hydroxyquinoline (0.324 mmol) dissolved in 2 mL acetonitrile is then added to the above mixture. The round bottom flask is then fitted with a reflux condenser and immersed in a oil bath at 90° C. The mixture is refluxed for 30 minutes after which it is cooled and the acetonitrile evaporated in vacuo. The crude residue obtained is purified by silica gel chromatography to obtain 2-(2-(4-fluorophenyl)ethyl)-3-methyl-4-hydroxyquinoline as the purified product.

### Synthesis of 2-(2-(4-[¹⁸F]-fluorophenyl)ethyl)-3-methyl-4-hydroxyquinoline

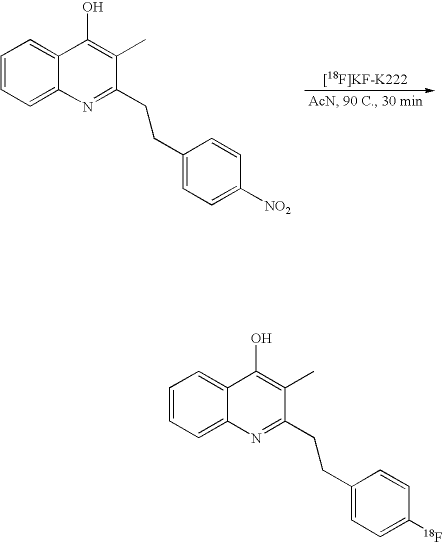

To a reaction vial containing 500 mCi of ¹⁸F in 350 mg of ¹⁸O water is added a 1 mL solution consisting of 10 mg of Kryptofix, 1 mg potassium carbonate, 0.005 mL water, and 0.95 mL acetonitrile. The vial is heated to remove all the solvents and dry acetonitrile (1 mL) is added to the vial. This is also removed by evaporation. 2-(2-(4-Nitrophenyl)ethyl)-3-methyl-4-hydroxyquinoline (5 mg) in acetonitrile is then added. The vial is sealed and heated for 30 minutes at 100° C. The mixture is diluted with dichloromethane and passed through a chromatography cartridge and eluted with tetrahydrofuran. The solvent is evaporated to provide the desired product.

### EXAMPLE 5

### Synthesis of 2-(2-(4-(2-[¹⁸F]-fluoroethoxy)phenyl)ethyl-3-methyl-4-hydroxyquinoline

### Synthesis of 2-(2-(4-methoxyphenyl)ethyl)-3-methyl-4-hydroxyquinoline

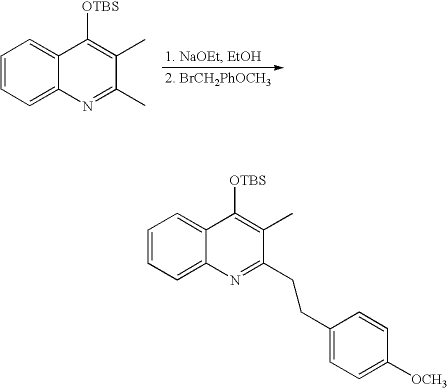

To 2,3-dimethyl-4-tert-butyldimethylsilyloxy quinoline (1.741 mmol) synthesized as in example 4 is added anhydrous ethanol (5 mL) followed by addition of sodium ethoxide (2.089 mmol). The above mixture is stirred for 10 minutes after which 4-methoxy benzylbromide (2.611 mmol) is added to it. The reaction mixture is stirred for 5 hours after which water is slowly added to the reaction mixture. The contents of the flask are then poured into a separatory funnel and the solution extracted with methylene chloride. The organic layer is then washed with water and brine and dried over MgSO₄. Purification using silica gel chromatography (Hexanes:Ethylacetate) provideed pure 2-(2-(4-methoxyphenyl)ethyl)-3-methyl-4-tert-butyldimethylsilyloxy quinoline.

### Synthesis of 2-(2-(4-hydroxyphenyl)ethyl)-3-methyl-4-tertbutydimethylsilyloxy quinoline

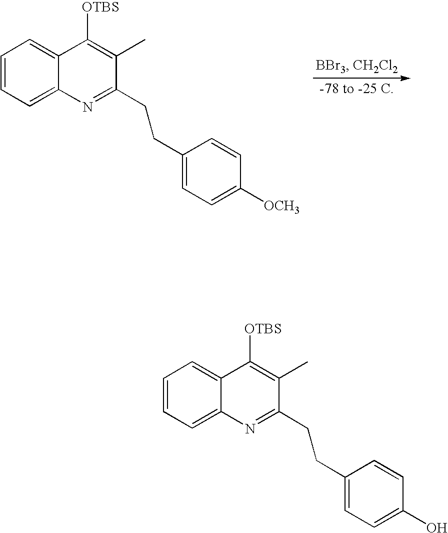

2-(2-(4-Methoxyphenyl)ethyl)-3-methyl-4-tert-butyldimethylsilyloxyquinoline (0.613 mmol) is dissolved in 5 mL methylene chloride and the solution is cooled to −78° C. Boron tribromide is then added to the above mixture by syringe (1.65 mmol) and the mixture is stirred for 1 hour. After this it is allowed to warm to −25° C. and stirred for an additional 1 hour. Saturated sodium hydrogen carbonate is added to the mixture to quench the boron tribromide. The mixture is then poured in a separatory funnel and extracted with methylene chloride, washed with water and brine, dried over MgSO₄, filtered, and concentrated. Purification of the crude mixture by silica gel chromatography provides the desired product.

### Synthesis of 2-(2-(4-(2-fluoroethoxy)phenyl)ethyl)-3-methyl-4-tert-butyldimethylsilyloxy quinoline

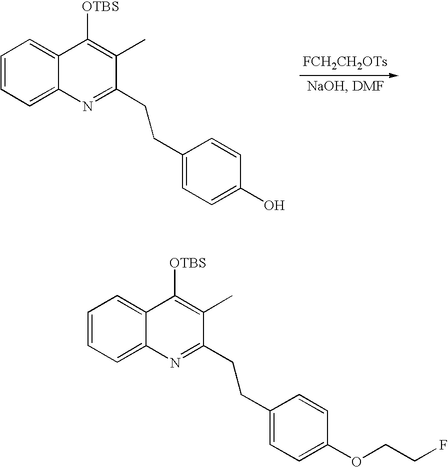

A 5 mL round bottom flask is charged with 2-(2-(4-hydroxyphenyl)ethyl)-3-methyl-4-tert-butyldimethylsilyloxyquinoline (0.254 mmol) and to it is added 2 mL of anhydrous DMF. This is followed by the addition of sodium hydroxide solution (0.254 mmol) and fluoroethyl tosylate (0.254 mmol). The reaction mixture is then immersed in an oil bath preheated to 80° C. and the reaction is allowed to stir for 1 hour. The mixture is then cooled to room temperature and the DMF is removed in vacuo. The residue obtained is dissolved in ethylacetate and washed with water and brine, dried over MgSO₄, filtered, and concentrated. The crude residue obtained after concentrating the organic solvent is purified by silica gel chromatography to obtain 2-(2-(4-(2-fluoroethoxy)phenyl)ethyl)-3-methyl-4-tert-butyldimethylsilyloxy quinoline as the purified product.

### Synthesis of 2-(2-(4-(2-fluoroethoxy)phenyl)ethyl)-3-methyl-4-hydroxyquinoline

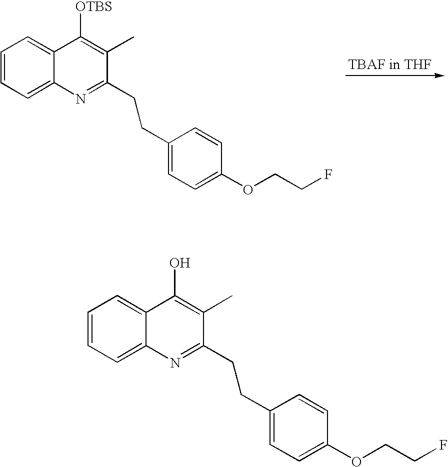

To a round bottom flask charged with 2-(2-(4-(2-fluoroethoxy)phenyl)ethyl)-3-methyl-4-tert-butyldimethylsilyloxy quinoline (0.113 mmol) is added tetrabutylammonium fluoride solution is THF (1M, 0.223 mmol) in ethanol solution. The above solution is then stirred for 30 minutes after which it is poured into a separatory funnel and extracted with methylene chloride. The organic layer is then washed with water and then with brine. It is then dried over MgSO₄ and filtered. The solvent is then removed in vacuo and the crude product obtained is purified using a short plug of silica gel using to obtain 2-(2-(4-(2-fluoroethoxy)phenyl)ethyl)-3-methyl-4-hydroxyquinoline as the purified product.

### Synthesis of 2-(2-(4-(2-[¹⁸F]-fluoroethoxy)phenyl)ethyl)-3-methyl-4-hydroxyquinoline

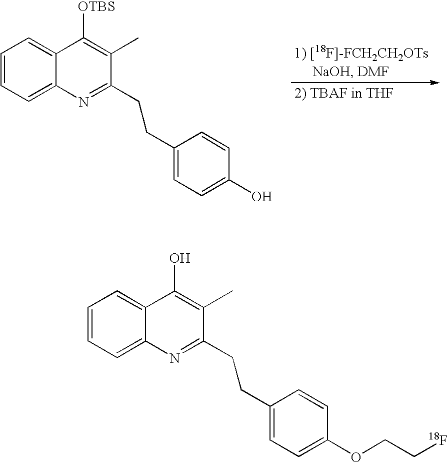

To a 3 mL conical reaction vial charged with 2-(2-(4-(2-fluoroethoxy)phenyl)ethyl)-3-methyl-4-tert-butyldimethylsilyloxyquinoline (5 mg, 0.0127 mmol) is added 0.3 mL of anhydrous DMF. This is followed by the addition of sodium hydroxide solution (5N, 2.9 μL, 0.0145 mmol). The solution is heated to 90° C. for 5 minutes after which a solution of 2-[¹⁸F]fluoroethyltosylate (prepared according to the procedure described in J. Labelled Compds. Radiopharm. 2001, 44, 627-642; 300-600 MBq) in DMF (300 μL) is added and the resulting mixture is stirred for 5 minutes at 90° C. A solution of tetrabutylammonium fluoride in THF (0.0254 mmol) is then added to the above mixture and this is stirred for 10 minutes at 90° C. The solution is then loaded on to an HPLC column followed by a solid phase column to elute the product in methanol.

### EXAMPLE 6

### Synthesis of 2-(2-(4-(5-[¹⁸F]-fluoropentyl)phenyl)ethyl)-3-methyl-4-hydroxyquinoline

### Synthesis of 5-(4-bromomethylphenyl)-5-oxomethylpentanoate

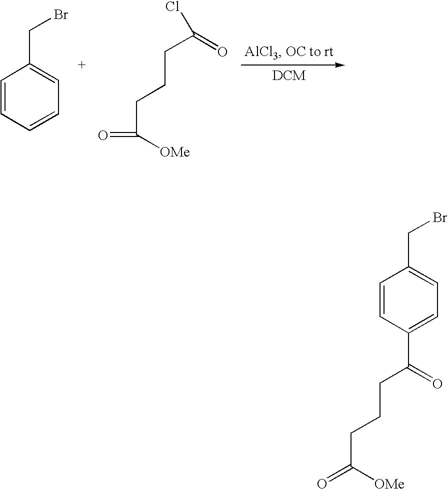

Benzyl bromide (5.883 mmol) dissolved in 7 mL anhydrous methylene chloride is added to a solution of 5-chloro-5-oxomethylpentanoate (5.883 mmol) and aluminum chloride (17.65 mmol) in methylene chloride at 0° C. The reaction mixture is stirred for 3 hours during which time the bath is allowed to warm to room temperature. Water is then slowly added to the reaction mixture to destroy excess aluminum chloride. The reaction mixture is then poured into a separatory funnel and extracted with methylene chloride. The organic layer is then washed with water and then with brine and is then dried over MgSO₄ and filtered. Purification is done using silica gel chromatography to obtain the desired product.

### Synthesis of 5-(4-bromomethylphenyl)methylpentanoate

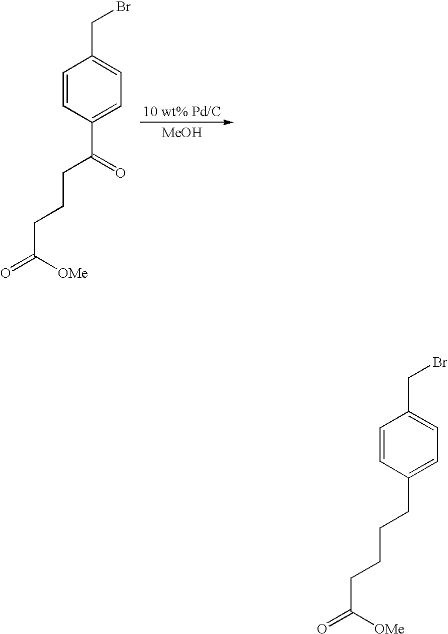

5-(4-(2-Bromoethyl)phenyl)-5-oxomethylpentanoate (1.677 mmol) is dissolved in 7 mL methanol and to it is added 10 wt % Pd/C (10 wt % relative to substrate). A balloon filled with hydrogen gas is applied to the flask and the reaction mixture is stirred for 12 hours. The flask is vented and the mixture is filtered through a pad of diatomaceous earth (Celite®) to obtain 5-(4-bromomethylphenyl)methyl pentanoate as the purified compound.

### Synthesis of 2-(2-(4-(5-methoxycarbonylpentyl)phenyl)ethyl)-3-methyl-4-tert-butyldimethylsilyloxy quinoline

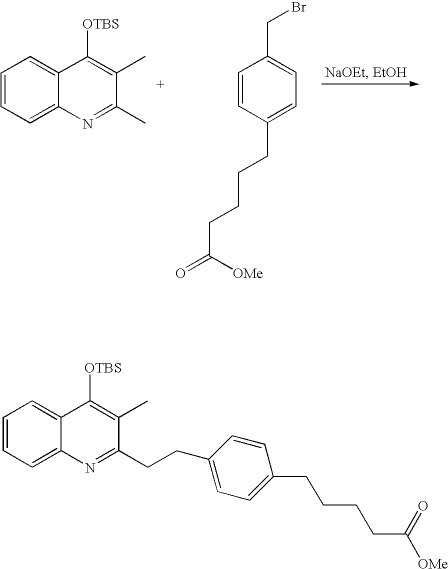

To 2,3-dimethyl-4-tert-butyldimethylsilyloxyquinoline (1.173 mmol; synthesized as in Example 4) is added anhydrous ethanol (5 mL) followed by addition of sodium ethoxide (1.408 mmol). The above mixture is stirred for 10 minutes after which 5-(4-bromomethylphenyl)methylpentanoate (1.76 mmol) is added. The reaction mixture is stirred for 5 hours after which water is slowly added to the reaction mixture. The contents of the flask are then poured into a separatory funnel and the solution is extracted with methylene chloride. The organic layer is then washed with water and brine and dried over MgSO₄ and filtered. Purification using silica gel chromatography provides the purified product.

### Synthesis of 2-(2-(4-(5-hydroxypentyl)phenyl)ethyl)-3-methyl-4-tert-butyldimethylsilyloxy quinoline

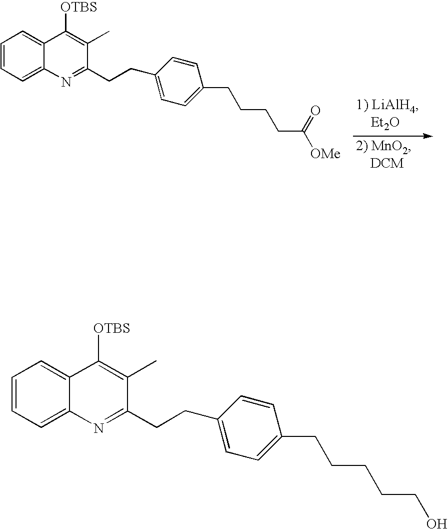

A 25 mL round bottom flask is charged with 2-(2-(4-(5-methoxycarbonylpentyl)phenyl)ethyl)-3-methyl-4-methoxyoxyquinoline (0.61 mmol) and to it is added anhydrous diethyl ether (5 mL). Lithium aluminum hydride (1.22 mmol) is then added to the above solution and the mixture is stirred for 3 hours. Water (1 mL for every mg of LiAlH₄), 15% NaOH (1 mL for every mg of LiAlH₄) followed by water (3 mL for every mg of LiAlH₄) is then added to the above mixture to destroy the lithium aluminum hydride and this is stirred for 20 minutes. The grainy precipitate formed is filtered and the filtrate is concentrated to provide 2-(2-(4-(5-hydroxypentyl)phenyl)ethyl)-3-methyl-4-tert-butyldimethylsilyloxy 2H-quinoline. The crude product obtained from the above reaction is then dissolved in methylene chloride and to it is added manganese dioxide (6.1 mmol). The reaction mixture is stirred for 4 hours after which it is filtered to obtain 2-(2-(4-(5-hydroxypentyl)phenyl)ethyl)-3-methyl-4-tert-butyldimethylsilyloxyquinoline as the purified product.

### Synthesis of 2-(2-(4-(5-tosyloxypentyl)phenyl)ethyl)-3-methyl-4-tert-butyldimethylsilyloxy quinoline

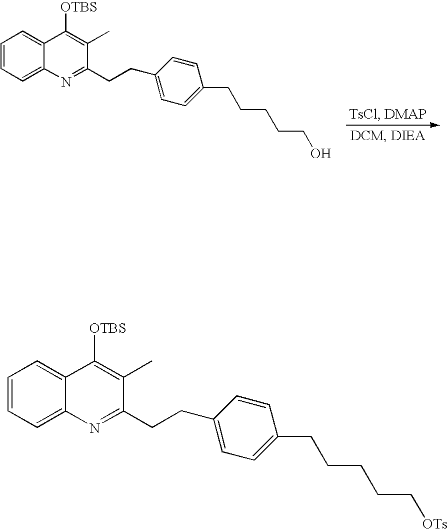

2-(2-(4-(5-Hydroxypentyl)phenyl)ethyl)-3-methyl-4-tert-butyldimethylsilyloxyquinoline (0.49 mmol) is charged to a round bottom flask and to it is added 5 mL methylene chloride. Dimethylaminopyridine (0.58 mmol), p-toluenesulfonyl chloride (0.58 mmol) and diisopropylethylamine (2.45 mmol, 0.43 mL) are then added to the flask and the reaction mixture is stirred for 4 hours. The mixture is then poured into a separatory funnel and water is added. The layers are separated and the organic layer is washed with brine and dried over MgSO₄ and filtered. The crude residue obtained after removing the organic solvent in vacuo is purified by silica gel chromatography to provide 2-(2-(4-(5-tosyloxypentyl)phenyl)ethyl)-3-methyl-4-tert-butyldimethylsilyloxyquinoline as the purified product.

### Synthesis of 2-(2-(4-(5-tosyloxypentyl)phenyl)ethyl)-3-methyl-4-hydoxyquinoline

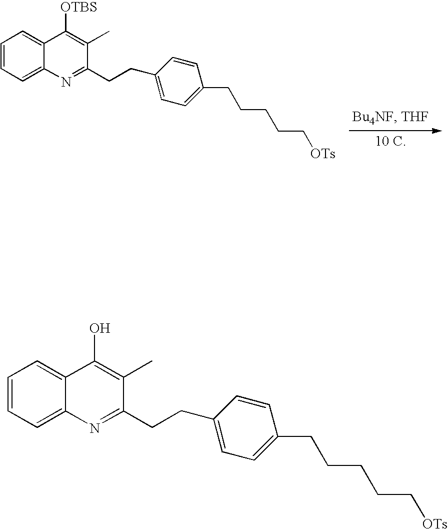

2-(2-(4-(5-Tosyloxypentyl)phenyl)ethyl)-3-methyl-4-tert-butyldimethylsilyloxy quinoline (0.405 mmol) is dissolved in a 15 mL round bottom flask and the flask is cooled to 110° C. Tetrabutylammonium fluoride solution (1.216 mmol, 1M in THF) is then added dropwise to the above solution while maintaining the reaction temperature at 10° C. The mixture is stirred for 2 hours after which all the solvent is removed in vacuo and the crude is purified using a short plug of silica to obtain 2-(2-(4-(5-tosyloxypentyl)phenyl)ethyl)-3-methyl-4-hydoxyquinoline as the purified product.

### Synthesis of 2-(2-(4-(5-fluoropentyl)phenyl)ethyl)-3-methyl-4-hydoxy quinoline

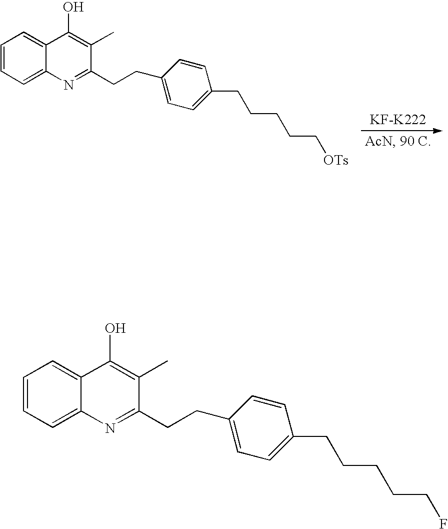

To a solution of potassium fluoride and Kryptofix 222 in 2 mL acetonitrile (0.0993 mmol each) is added a solution of 2-(2-(4-(5-tosyloxypentyl)phenyl)ethyl)-3-methyl-4-hydoxyquinoline (0.0993 mmol) in 11 mL acetonitrile. The flask is then fitted with a reflux condenser and the solution is immersed in an oil bath preheated to 90° C. and refluxed for 30 minutes. The solution is then cooled to room temperature and the contents concentrated on a rotary evaporator. The crude mixture obtained is subjected to silica gel chromatography to obtain the desired compound.

### Synthesis of 2-(2-(4-(5-[¹⁸F]-fluoropentyl)phenyl)ethyl)-3-methyl-4-hydoxy quinoline

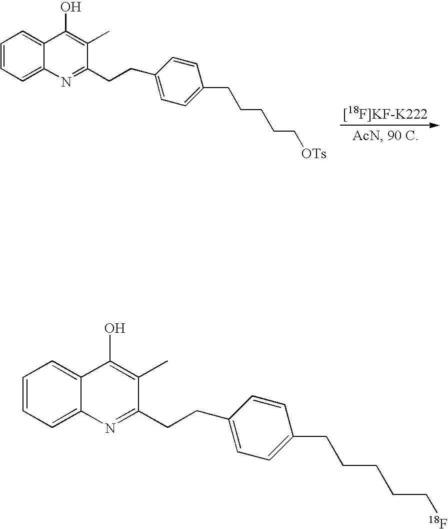

To a 5 mL reaction vial containing 500 mCi of ¹⁸F in 350 mg of ¹⁸O water is added a 1 mL solution consisting of 10 mg of Kryptofix 222, 1 mg potassium carbonate, 0.005 mL water and 0.95 mL acetonitrile. The vial is heated to remove all the solvents and dry acetonitrile (1 mL) is added to the vial. This is also removed by evaporation. 2-(2-(4-(5-Tosyloxypentyl)phenyl)ethyl)-3-methyl-4-hydoxyquinoline (5 mg) in acetonitrile is then added. The vial is sealed and heated for 30 minutes at 100° C. The mixture is diluted with dichloromethane and passed through a chromatography cartridge and eluted with tetrahydrofuran. The solvent is evaporated to provide the desired product.

It will be evident to one skilled in the art that the present disclosure is not limited to the foregoing illustrative examples, and that it can be embodied in other specific forms without departing from the essential attributes thereof. It is therefore desired that the examples be considered in all respects as illustrative and not restrictive, reference being made to the appended claims, rather than to the foregoing examples, and all changes which come within the meaning and range of equivalency of the claims are therefore intended to be embraced therein.

What is claimed is:

1. A compound of formula (I) 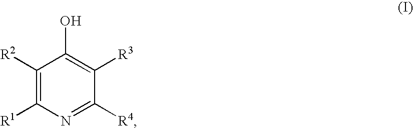
or a pharmaceutically acceptable salt thereof, wherein
R¹ and R² are alkoxy optionally substituted with an imaging moiety; or
R¹ and R², together with the carbon atoms to which they are attached, form a six-membered aromatic ring containing zero or one nitrogen atoms optionally substituted with alkoxy or an imaging moiety; wherein the alkoxy is further optionally substituted with an imaging moiety; and
R³ and R⁴ are independently alkenyl, alkyl, alkynyl, aryloxyalkyl, or arylalkyl, wherein the alkenyl, the alkyl, the alkynyl, and the alkyl part of the aryloxyalkyl and the arylalkyl are each optionally substituted with an imaging moiety, and wherein the aryl part of the aryloxyalkyl and the arylalkyl are optionally substituted with alkoxy, a second alkyl group, or an imaging moiety, wherein the alkoxy and the second alkyl group are each optionally substituted with an imaging moiety;
provided that at least one imaging moiety is present.

2. The compound of claim 1 wherein the imaging moiety is selected from a radioisotope for nuclear medicine imaging, a paramagnetic species for use in MRI imaging, an echogenic entity for use in ultrasound imaging, a fluorescent entity for use in fluorescence imaging, and a light-active entity for use in optical imaging.

3. The compound of claim 2 wherein the imaging moiety is a paramagnetic species for use in MRI imaging, wherein the paramagnetic species is selected from Gd³⁺, Fe³⁺, In³⁺, and Mn²⁺.

4. The compound of claim 2 wherein the imaging moiety is an echogenic entity for use in ultrasound imaging, wherein the echogenic entity is a fluorocarbon encapsulated surfactant microsphere.

5. The compound of claim 2 wherein the imaging moiety is a radioisotope for nuclear medicine imaging wherein the radioisotope is selected from ¹¹C, ¹³N, ¹⁸F, ¹²³I, ¹²⁵I, ^{99m}Tc, ⁹⁵Tc, ¹¹¹In, ⁶²Cu, ⁶⁴Cu, ⁶⁷Ga, and ⁶⁸Ga.

6. The compound of claim 5 wherein the radioisotope is ¹⁸F.

7. The compound of claim 5 wherein the radioisotope is ^{99m}Tc.

8. An imaging agent comprising a compound of formula (I) and a metal bonding unit having a formula selected from 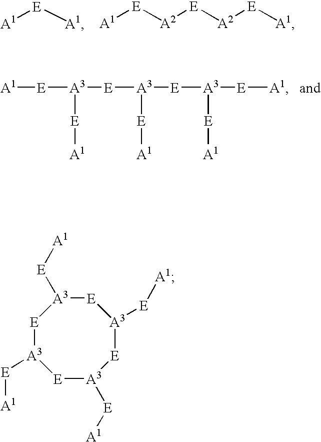
wherein
each A¹ is independently selected from a bond to the compound of formula (I), —NR⁵R⁶, —SH, —S(Pg), —OH, —PR⁵R⁶, and —P(O)R⁷R⁸;
each A² is independently selected from S, O, and PR²
A³ is N;
each E is independently selected from C₁₋₁₀alkylene substituted with 0-3 R⁹ groups, C₆₋₁₀arylene substituted with 0-3 R⁹ groups, C₃₋₁₀cycloalkylene substituted with 0-3 R⁹ groups, heterocyclyl-C₁₋₁₀alkylene substituted with 0-3 R⁹ groups, C₆₋₁₀aryl-C₁₋₁₀alkylene substituted with 0-3 R⁹ groups, and heterocyclyl substituted with 0-3 R⁹ groups;
R⁵ and R⁶ are each independently selected from a bond to the compound of formula (I), C₁₋₁₀alkyl substituted with 0-3 R⁸ groups, C₆₋₁₀aryl substituted with 0-3 R⁸ groups, C₃₋₁₀cycloalkyl substituted with 0-3 R⁸ groups, heterocyclyl-C₁₋₁₀alkyl substituted with 0-3 R⁸ groups, and heterocyclyl substituted with 0-3 R⁸ groups;
R⁷ and R⁸ are each independently selected from a bond to the compound of formula (I), C₁₋₁₀alkyl substituted with 0-3 R⁹ groups, C₆₋₄₀aryl substituted with 0-3 R⁹ groups, C₃₋₁₀cycloalkyl substituted with 0-3 R⁹ groups, heterocyclyl-C₁₋₁₀alkyl substituted with 0-3 R⁹ groups, C₆₋₁₀aryl-C₁₋₁₀alkyl substituted with 0-3 R⁹ groups, heterocyclyl substituted with 0-3 R⁹ groups, and hydroxy;
each R⁹ is independently selected from a bond to the compound of formula (I), C₂₋₄alkenyl, C₁₋₆alkoxy, C₁₋₆alkoxycarbonyl, di(C₁₋₆alkyl)amino, C₁₋₆alkylcarbonyl, amino, cyano, C₃₋₆cycloalkyl, formyl, halo, haloalkoxy, haloalkyl, hydroxy, nitro, and oxo; and
Pg is a thiol protecting group;
provided at least one bond to the compound of formula (I) is present.

9. A complex comprising a compound of formula (I) and a metal bonding unit having a formula selected from 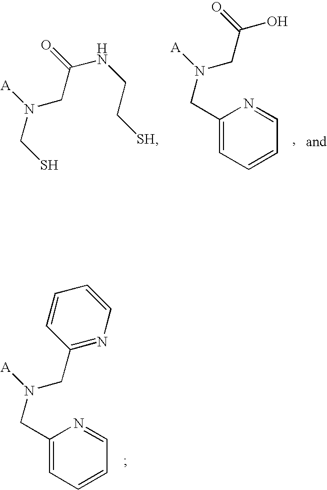
wherein A is a bond to the compound of formula (I).

10. The complex of claim 9 wherein the imaging moiety is ^{99m}Tc.

11. A composition comprising a compound of claim 1 and a pharmaceutically acceptable carrier.

12. A method of imaging myocardial perfusion in a patient, the method comprising:
(a) administering to a patient a compound of formula (I) 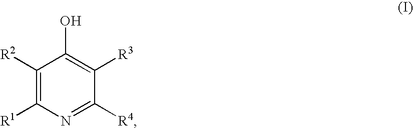
or a pharmaceutically acceptable salt thereof, wherein
R¹ and R² are alkoxy optionally substituted with an imaging moiety; or
R¹ and R², together with the carbon atoms to which they are attached, form a six-membered aromatic ring containing zero or one nitrogen atoms optionally substituted with alkoxy or an imaging moiety; wherein the alkoxy is further optionally substituted with an imaging moiety; and
R³ and R⁴ are independently alkenyl, alkyl, alkynyl, aryloxyalkyl, or arylalkyl, wherein the alkenyl, the alkyl, the alkynyl, and the alkyl part of the aryloxyalkyl and the arylalkyl are each optionally substituted with an imaging moiety, and wherein the aryl part of the aryloxyalkyl and the arylalkyl are optionally substituted with alkoxy, a second alkyl group, or an imaging moiety, wherein the alkoxy and the second alkyl group are each optionally substituted with an imaging moiety;
provided that at least one imaging moiety is present; and
(b) acquiring an image of a site of concentration of the compound in the patient by a diagnostic imaging technique.

13. The method of claim 12 wherein the imaging moiety is a radioisotope for nuclear medicine imaging, a paramagnetic species for use in MRI imaging, an echogenic entity for use in ultrasound imaging, a fluorescent entity for use in fluorescence imaging, or a light-active entity for use in optical imaging.

14. The method of claim 13 wherein the imaging moiety is a paramagnetic species for use in MRI imaging, wherein the paramagnetic species is selected from Gd³⁺, Fe³⁺, In³⁺, and Mn²⁺.

15. The method of claim 13 wherein the imaging moiety is an echogenic entity for use in ultrasound imaging, wherein the echogenic entity is a fluorocarbon encapsulated surfactant microsphere.

16. The method of claim 13 wherein the imaging moiety is a radioisotope for nuclear medicine imaging wherein the radioisotope is selected from ¹¹C, ¹³N, ¹⁸F, ¹²³I, ¹²⁵I, ^{99m}Tc, ⁹⁵Tc, ¹¹¹In, ⁶²Cu, ⁶⁴Cu, ⁶⁷Ga, and ⁶⁸Ga.

17. The method of claim 16 wherein the radioisotope is ¹⁸F.

18. The method of claim 16 wherein the radioisotope is ^{99m}Tc.
